(19) United States
(12) Patent Application Publication
(10) Pub. No.: US 2018/0256413 A1
(43) Pub. Date: Sep. 13, 2018

(54) Menstrual underswear

(71) Applicant: Prundisc, LLC, Brooklyn Park, MN (US)

(72) Inventor: Catherine Apeatro, Brooklyn Park, MN (US)

(21) Appl. No.: 15/454,015

(22) Filed: Mar. 9, 2017

(51) Int. Cl. A61F 13/476; A61F 13/58; A61F 13/472

(52) U.S. Cl. CPC A61F 13/476; A61F 13/472; A61F 13/58

(57) ABSTRACT

The disclosed embodiments illustrate an item of underwear. The item of underwear includes a body portion worn around a lower torso. The item of underwear further includes a first leg portion, and a second leg portion. A crotch portion is disposed below the body portion. A first aperture is disposed between the crotch portion and the first leg portion. A first menstrual pad bonding surface is disposed on an external surface of the crotch portion adjacent to the first aperture. A second aperture is disposed between the crotch portion and the second leg portion. A second menstrual pad bonding surface is disposed on an external surface of the crotch portion adjacent to the second aperture.

### TECHNICAL FIELD

The presently disclosed embodiments are related, in general, to undergarments such as underwear. More particularly, the presently disclosed embodiments are related to an item of the underwear for securing a menstrual pad.

### BACKGROUND

Undergarments such as underwear are items of clothing that are worn by many people, including female users. The undergarments are constructed in various types and styles for providing comfort and style to the female users. Many styles of underwear include seams. A seam is generally formed at a junction of two or more pieces of material. Depending on the style of the underwear, seems are visible through the outer garments of the wearer, and are unsightly.

Many female underwear users prefer to purchase underwear comprised of elastic materials. Elastic materials remove unsightly abdominal fat and contour the outlines of the wearer's body. Typically the elastic materials will be employed in both the abdominal and about the thighs of the wearer.

Further, various products such as tampons, menstrual pads, and other sanitary napkins are available to women to manage menstrual flow. Menstrual pads are typically secured to underwear by means of wings or other securing means. Currently, if a wearer of elastic and seamless underwear were to choose a menstrual pad to manager her flow there is no way to secure the menstrual pad firmly in place. The result of failing to secure a menstrual pad in place is leakage, soiling of clothing, and discomfort. Therefore there is the current need for an undergarment and systems that may conceal bulges of fat, be seamless, and secure a menstrual pad.

### SUMMARY

It will be understood that this disclosure is not limited to the particular systems, and methods described, as there can be multiple possible embodiments of the present disclosure which are not expressly illustrated in the present disclosure. It is also to be understood that the terminology used in the description is for the purpose of describing the particular versions or embodiments only, and is not intended to limit the scope of the present disclosure.

According to embodiments illustrated herein, there is provided an item of underwear. The item of underwear includes a body portion that is worn around a lower torso. The item of underwear further includes a first leg portion, and a second leg portion. A crotch portion is disposed below the body portion. A first aperture is disposed between the crotch portion and the first leg portion. A first menstrual pad bonding surface is disposed on an external surface of the crotch portion adjacent to the first aperture. A second aperture is disposed between the crotch portion and the second leg portion. A second menstrual pad bonding surface is disposed on an external surface of the crotch portion adjacent to the second aperture.

According to embodiments illustrated herein, there is provided a system for securing a menstrual pad. The system includes a body portion that is worn around a lower torso. The system further includes a first leg portion, and a second leg portion. A crotch portion is disposed below the body portion. A first aperture is disposed between the crotch portion and the first leg portion. A second aperture is disposed between the crotch portion and the second leg portion. A menstrual pad with wings is disposed between the crotch portion and the lower torso. The wings of the menstrual pad pass through the first aperture and the second aperture, and bond to an external surface of the crotch portion.

Other systems, methods, features and advantages will be, or will become, apparent to one with skill in the art upon examination of the following figures and detailed description. It is intended that all such additional systems, methods, features and advantages be included within this description, be within the scope of the embodiments, and be protected by the following claims and be defined by the following claims. Further aspects and advantages are discussed below in conjunction with the description.

### BRIEF DESCRIPTION OF DRAWINGS

The accompanying drawings illustrate the various embodiments of systems, methods, and other aspects of the disclosure. Any person with ordinary skills in the art will appreciate that the illustrated element boundaries (e.g., boxes, groups of boxes, or other shapes) in the figures represent one example of the boundaries. In some examples, one element may be designed as multiple elements, or multiple elements may be designed as one element. In some examples, an element shown as an internal component of one element may be implemented as an external component in another, and vice versa. Further, the elements may not be drawn to scale. Non-limiting and non-exhaustive descriptions are described with reference to the following drawings. The components in the figures are not necessarily to scale, emphasis instead being placed upon illustrating principles.

Various embodiments will hereinafter be described in accordance with the appended drawings, which are provided to illustrate and not to limit the scope in any manner, wherein similar designations denote similar elements, and in which:

FIG. 1 is a front view of a female user wearing an item of underwear, in accordance with the concepts of the present disclosure;

FIG. 2 is the item of underwear for securing a menstrual pad, in accordance with the concepts of the present disclosure;

FIG. 3 is an underside view of the item of underwear showing wings of the menstrual pad, in accordance with the concepts of the present disclosure;

FIG. 4 is an underside view of the item of underwear showing a first menstrual pad bonding surface and a second menstrual pad bonding surface, in accordance with the concepts of the present disclosure; and

FIG. 5 is the menstrual pad that may be used with the item of underwear, in accordance with the concepts of the present disclosure.

### DETAILED DESCRIPTION

The present disclosure is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

Some embodiments of this invention, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present invention, the preferred, systems and methods are now described.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

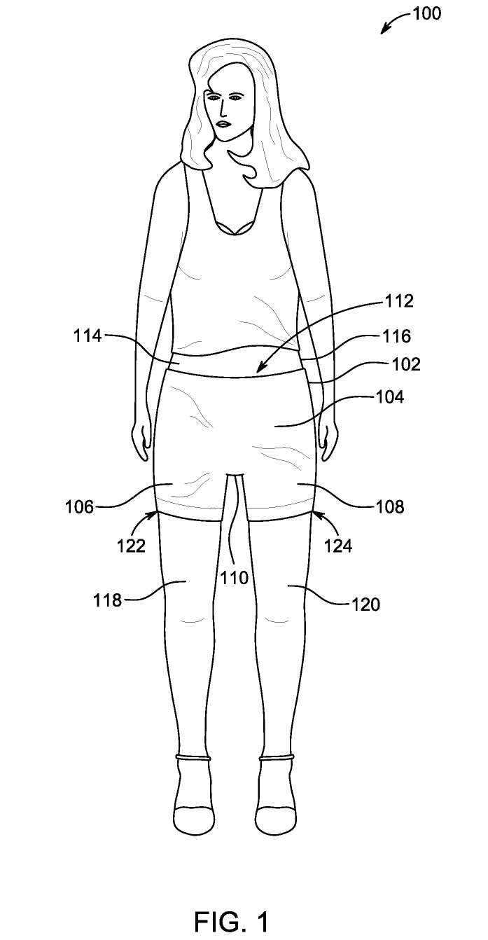

FIG. 1 is a front view of a female user 100 wearing an item of underwear 102, in accordance with the concepts of the present disclosure. The item of underwear 102 includes a body portion 104, a first leg portion 106, a second leg portion 108, and a crotch portion 110.

The item of underwear 102 is a particular piece of clothing that may be worn by the female user 100. It should be noted that the item of underwear 102 may be worn around a lower torso 112 of the female user 100. The item of underwear 102 may be designed in a particular type and/or a style for providing comfort to the female user 100 during a menstrual cycle. It should be noted that the menstrual cycle may lead to dreaded time for the female user 100, which has up and downs due to hormonal changes. Further, the item of underwear 102 may be designed such that the body portion 104 of the item of underwear 102, may be worn around the lower torso 112 of the female user 100. The body portion 104 may be disposed around an abdominal region 114 of the female user 100. In an embodiment, the item of underwear 102 may include an elastic waistband (not shown) that may be attached to an open top end of the body portion 104. The elastic waistband may be utilized to keep the open top end in a snug fit about a waist 116 of the female user 100.

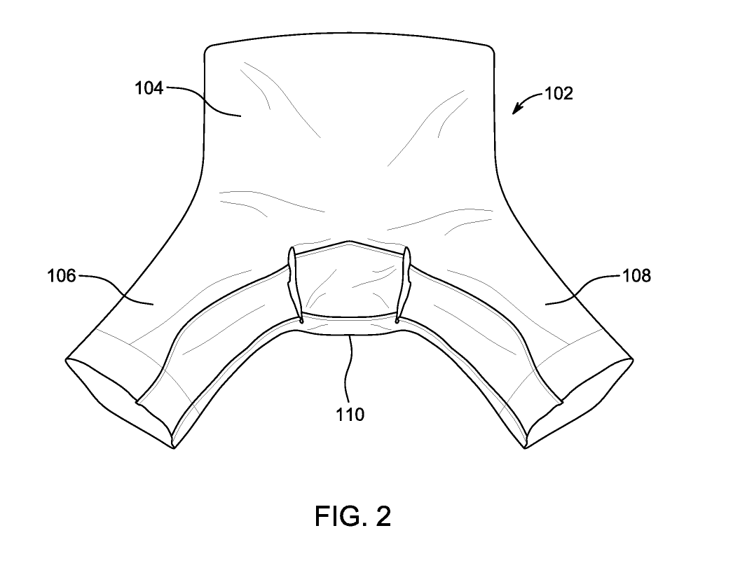

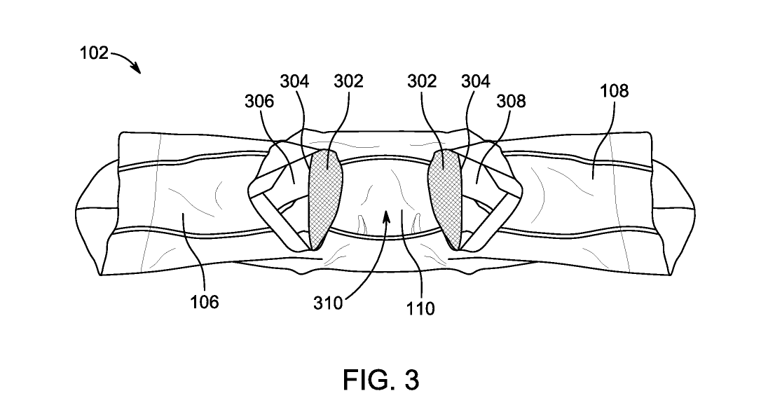

Further, the item of underwear 102 may include the first leg portion 106 for insertion of a first leg 118 of the female user 100. Similarly, the item of underwear 102 may include the second leg portion 108 for insertion of a second leg 120 of the female user 100. It should be noted that the first leg portion 106 and the second leg portion 108 may be disposed around a first thigh 122 and a second thigh 124 of the female user 100 respectively. The underwear 102 may be made up of a material such as an elastic material. It will be apparent to one skilled in the art that the above-mentioned material has been provided only for illustration purposes. The underwear 102 may be made up of some other material such as, but not limited to, non-woven material, as well, without departing from the scope of the disclosure. Referring to FIGS. 1 and 2, the item of underwear 102 may further include the crotch portion 110 which is disposed below the body portion 104. It should be noted that the crotch portion 110 may lie in between the first leg portion 106 and the second leg portion 108. The crotch portion 110 may include a structure (not shown) for holding a menstrual pad (not shown) within the crotch portion 110 in a proper position. The menstrual pad is an absorbent item that may be worn by the female user 100 while the female user 100 is menstruating. It should be noted that the item of underwear 102 may be provided with slits for securing the menstrual pad. The detailed description of the item of underwear 102 will be described later in conjunction with FIG. 3.

FIG. 3 is an underside view of the item of underwear 102 showing wings 302 of a menstrual pad 304, in accordance with the concepts of the present disclosure. The item of underwear 102 showing the wings 302 of the menstrual pad 304 is explained in conjunction with FIGS. 1, and 2.

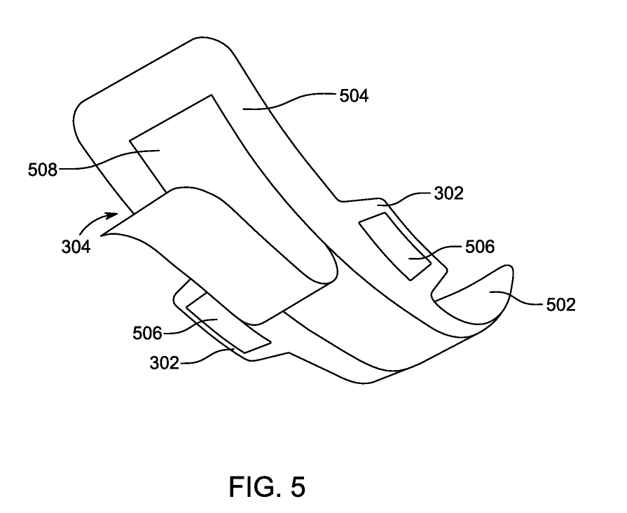

As discussed above, the item of underwear 102 may include the crotch portion 110 which is disposed below the body portion 104. The crotch portion 110 may include the structure (not shown) for holding the menstrual pad 304 within the crotch portion 110 in a proper position. It should be noted that the menstrual pad 304 is an absorbent item that may be worn by the female user 100 while the female user 100 is menstruating. The menstrual pad 304 with the wings 302 may be disposed between the crotch portion 110 and the lower torso 112. As shown in FIG. 3, the item of underwear 102 may include a first aperture 306 which is disposed between the crotch portion 110 and the first leg portion 106. Similarly, the item of underwear 102 may include a second aperture 308 that is disposed between the crotch portion 110 and the second leg portion 108. It should be noted that the first aperture 306 and the second aperture 308, may be utilized for inserting the wings 302 of the menstrual pad 304. The wings 302 of the menstrual pad 304 may be bonded, as an example, using adhesive, to an external surface 310 of the crotch portion 110. The detailed description of the menstrual pad 304 will be described later in conjunction with FIG. 5.

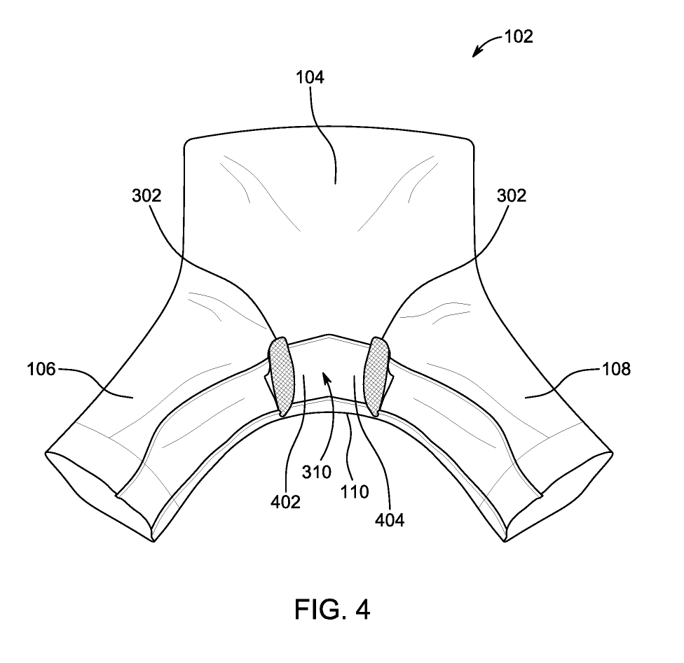

FIG. 4 is an underside view of the item of underwear 102 showing a first menstrual pad bonding surface 402 and a second menstrual pad bonding surface 404, in accordance with the concepts of the present disclosure. The item of underwear 102 showing the first menstrual pad bonding surface 402 and the second menstrual pad bonding surface 404 is explained in conjunction with FIGS. 1, 2, and 3.

As discussed above, the item of underwear 102 may include the first aperture 306 (shown in FIG. 3) that is disposed between the crotch portion 110 and the first leg portion 106. Similarly, the item of underwear 102 may include the second aperture 308 (shown in FIG. 3) that is disposed between the crotch portion 110 and the second leg portion 108. Further, the item of underwear 102 may include the first menstrual pad bonding surface 402 that is disposed on the external surface 310 of the crotch portion 110 adjacent to the first aperture 306. Similarly, the item of underwear 102 may include the second menstrual pad bonding surface 404 that is disposed on the external surface 310 of the crotch portion 110 adjacent to the second aperture 308. It should be noted that the first menstrual pad bonding surface 402 and the second menstrual pad bonding surface 404 may be bonded to the external surface 310 of the crotch portion 110.

It will be apparent to one skilled in the art that the underwear 102 may be available in small, medium, large, and extra large sizes. Further, the underwear 102 may be produced in various colors with decorative appliques such as lace or bows, and designs such as flowers. It should be noted that the underwear 102 may be of different styles such as, but not limited to, pants, shorts, bikini and/or brief versions with different leg cuts, as well as a variety of colors and decorative designs, without departing from the scope of the disclosure.

FIG. 5 is the menstrual pad 304 that may be used with the item of underwear 102, in accordance with the concepts of the present disclosure. The menstrual pad 304 that may be used with the item of underwear 102 is explained in conjunction with FIGS. 1, 2, 3, and 4.

As shown in FIG. 5, the menstrual pad 304 may include the wings 302 which carry adhesive, and may be bonded to the external surface 310 of the crotch portion 110 of the item of underwear 102. The menstrual pad 304 may include an absorbent surface 502 and a backing 504 that may include adhesive. It should be noted that adhesive of the wings 302 and adhesive of the backing 504 may be protected by a protective sheet 506 and a protective sheet 508 respectively. It should be noted that the menstrual pad 304 may be used to absorb the menstrual flow or discharge.

Further, the menstrual pad 304 may be of different types such as disposable menstrual pads, or cloth menstrual pads. The disposable menstrual pads may be of different types such as ultra-thin, or maxi/super. On the other hand, the cloth menstrual pads may be washable or reusable pads. The menstrual pad 304 may be made up of materials such as, but not limited to, bleached rayon, or cotton. Further, the menstrual pad 304 may include fragrance, and antibacterial agents. It should be noted that the menstrual pad 304 may also be referred as a sanitary pad, a sanitary napkin, a sanitary towel, or a pad. It will be apparent to one skilled in the art that the menstrual pad 304 mentioned above has been provided only for illustration purposes, without departing from the scope of the disclosure.

It will be apparent to one skilled that the above mentioned description of the item of underwear 102 has been provided only for illustration purposes. As discussed above, the item of underwear 102 may include the body portion 104, the first leg portion 106, the second leg portion 108, and the crotch portion 110. The item of underwear 102 may include some other portions as well, without departing from the scope of the disclosure.

The disclosed embodiments encompass numerous advantages. Various embodiments of an item of underwear are disclosed. The disclosure discloses that the item of underwear with slits for securing a menstrual pad. The disclosure discloses that the item of underwear includes a first aperture which is disposed between a crotch portion and a first leg portion. Similarly, the item of underwear includes a second aperture that is disposed between the crotch portion and a second leg portion. It should be noted that the first aperture and the second aperture, may be utilized for inserting wings of the menstrual pad. The wings of the menstrual pad may be bonded to an external surface of the crotch portion, and thus results in securing the menstrual pad in place so that the menstrual pad does not move. Further, the disclosure discloses that the item of underwear may include the menstrual pad with the wings that may fit snugly against a first thigh and a second thigh of a first leg and a second leg of the female user respectively.

Further, the disclosure discloses that the item of underwear removes unsightly visible underwear seams. Also, the item of underwear is configured to smooth and conceal bulges of abdominal fat. The disclosure further discloses that the item of underwear may be worn by the female user during a menstrual flow or other discharge without fear of spoiling expensive clothing. Also, the item of the underwear eliminates leakage, and/or stains on the underwear and outer wear. Thus, such type of the item of the underwear provides comfort to the female user during the menstrual cycle.

Various embodiments of an item of underwear with slits for securing a menstrual pad have been disclosed. However, it should be apparent to those skilled in the art that modifications, in addition to those described, are possible without departing from the inventive concepts herein. The embodiments, therefore, are not restrictive, except in the spirit of the disclosure. Moreover, in interpreting the disclosure, all terms should be understood in the broadest possible manner consistent with the context. In particular, the terms “comprises” and “comprising” should be interpreted as referring to elements, components, or steps, in a non-exclusive manner, indicating that the referenced elements, components, or steps may be present, used, or combined with other elements, components, or steps that are not expressly referenced.

A person with ordinary skills in the art will appreciate that the systems, modules, and sub-modules have been illustrated and explained to serve as examples and should not be considered limiting in any manner. It will be further appreciated that the variants of the above disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application.

The claims can encompass embodiments for hardware and software, or a combination thereof.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. An item of underwear comprising:
a body portion worn around a lower torso;
a first leg portion;
a second leg portion;
a crotch portion disposed below the body portion;
a first aperture disposed between the crotch portion and the first leg portion;
a first menstrual pad bonding surface disposed on an external surface of the crotch portion adjacent to the first aperture;
a second aperture disposed between the crotch portion and the second leg portion; and
a second menstrual pad bonding surface disposed on an external surface of the crotch portion adjacent to the second aperture.

2. The item of underwear as recited in claim 1, wherein the underwear is comprised of an elastic material.

3. The item of underwear as recited in claim 1, wherein the body portion is disposed around an abdominal region.

4. The item of underwear as recited in claim 1, wherein the first leg portion is disposed around a first thigh and the second leg portion is disposed around a second thigh.

5. A system for securing a menstrual pad comprising:
a body portion worn around a lower torso;
a first leg portion;
a second leg portion;
a crotch portion disposed below the body portion;
a first aperture disposed between the crotch portion and the first leg portion;
a second aperture disposed between the crotch portion and the second leg portion; and
the menstrual pad with wings disposed between the crotch portion and the lower torso, wherein the wings of the menstrual pad pass through the first and second apertures and bond to an external surface of the crotch portion.
